(19) United States
(12) Patent Application Publication
(10) Pub. No.: US 2010/0003740 A1
(43) Pub. Date: Jan. 7, 2010

(54) CHEMICALLY DEFINED CULTURE MEDIUM FOR NEISSERIA

(75) Inventors: Jim Wade, Wiltshire (GB); Michelle Graver, Wiltshire (GB)

Correspondence Address: EVAN LAW GROUP LLC, 600 WEST JACKSON BLVD., SUITE 625, CHICAGO, IL, 60661, US

(73) Assignee: HEALTH PROTECTION AGENCY, WILTSHIRE (GB)

(21) Appl. No.: 12/301,594

(22) PCT Filed: May 18, 2007
(86) PCT No.: PCT/GB2007/001831

§ 371 Date: Jun. 24, 2009

(30) Foreign Application Priority Data
May 19, 2006 (GB) 0610009.3

(51) Int. Cl. C12N 1/20

(52) U.S. Cl. 435/253.6

(57) ABSTRACT

There is provided a chemically-defined liquid culture medium comprising an energy source, a carbon source, a nitrogen source, an amino acid source, a purine synthesis source, a pyrimidine synthesis source and a buffer; characterised in that the culture medium has a pH less than 7, preferably less than 6.9, most preferably about 6.8.

The present invention relates to a chemically-defined liquid culture medium, and to uses thereof, including methods for culturing bacteria using said culture medium.

In order to grow in the laboratory, a microorganism such as a bacterium must be provided with the appropriate biochemical and biophysical environment, which is made available as a culture medium. Culture media include liquid media (broths) and solid media (such as agar media). Depending upon the special needs of a particular microorganism (eg. bacterium), a large variety and types of culture media have been developed with different purposes and uses.

Culture media may be categorised as “undefined/complex media” or “chemically defined media”, depending on their composition. Most bacteriological media are complex and undefined.

Examples of undefined (complex) media include rich solid media such as chocolate agar and blood agar, and broths such as nutrient broth, brain heart infusion and tryptone soya broth. Complex media contain components of biological origin such as blood, milk, serum, yeast/beef extract and/or tissue extracts.

Thus, a problem associated with undefined (complex) media is that their exact chemical composition is unknown. In this regard, complex media are a potential source of microbial inhibitors such as fatty acids. Complex media may also contain proteins, which limits their utility for growing bacteria for vaccine studies. A further disadvantage associated with most complex media is uncontrolled divalent cation concentrations. In addition, the pH of some commercially available complex media may be inappropriate to allow initiation of the growth of some bacteria, in particular some fastidious bacteria such as N. gonorrhoeae.

In contrast, a chemically-defined (synthetic) medium is one in which the exact chemical composition is known, and which is therefore consistently reproducible. Unlike complex media, chemically defined media contain substantially no (preferably no) infusates, extracts, digests or peptones. That said, defined media always contain a specific, defined, energy source, a carbon source and a nitrogen source. In addition, defined media typically contain selected concentrations of specific amino acid sources, purine/pyrimidine synthesis sources, vitamins and other micro-nutrients.

Also critical to successful growth of microorganisms in vitro is the provision of a permissive range of physical conditions such as divalent cation concentration, O₂ concentration, temperature and pH.

Fastidious bacteria are so-named because they are particularly difficult to cultivate in vitro, due to their very specific requirements—in particular, nutritional requirements or a tendency to autolysis. Examples of fastidious bacteria commonly encountered in clinical microbiology practice include Haemophilus influenzae, Streptococcus pneumoniae and Neisseria gonorrhoeae. Of these, N. gonorrhoeae is the most fastidious. Throughout the present specification, reference to N. gonorrhoeae embraces all fastidious bacteria. For convenience and to avoid repetition, we have cited N. gonorrhoeae as representative of all fastidious bacteria.

There is considerable variation in nutritional requirements between different strains—known as “auxotypes”—of N. gonorrhoeae. By way of example, some strains may require one or more of arginine, proline, hypoxanthine, uracil or ornithine. Other strains do not require any of these (identified) specific agents, and are thus referred to as prototrophic or non-requiring (NR) strains. Throughout the present specification, reference to N. gonorrhoeae embraces all auxotypes—of N. gonorrhoeae.

A problem commonly encountered in clinical microbiology is that very low concentrations of certain bacteria can be difficult to ‘resuscitate’ and multiply to high density, which makes downstream detection/identification and antibiotic susceptibility testing difficult (or even impossible). In this regard, in some situations—such as early in an infection, infection with a bacterium of low pathogenicity, or after antibiotic therapy—bacteria may be present in very low numbers. This is particularly the case with regard to fastidious bacteria, such as N. gonorrhoeae.

‘Enrichment broths’ may be used to facilitate detection of such bacteria in clinical materials, which are normally pathogen-free—eg. tissues removed at surgery and ‘sterile fluids’ such as blood, cerebrospinal fluid, joint fluid, ascites and aqueous humour. The presence of any bacteria in such specimens is abnormal and therefore sought by enrichment. However, currently there is no enrichment broth available that will reliably recover very low numbers of fastidious bacteria such as N. gonorrhoeae from such specimens. In the case of disseminated N. gonorrhoeae infection, bacteria may be present in synovial fluid, cerebrospinal fluid and blood.

Generally, culture of fastidious bacteria such as N. gonorrhoeae relies on the use of very rich, complex, solid media (such as chocolate agar) or undefined rich broths incorporating infusates, extracts or digests (for example, nutrient broth, brain heart infusions).

However, these rich media are undefined and, as discussed above, suffer from a number of disadvantages, such as the presence of inhibitors and proteins, and lack consistent reproducibility.

Furthermore, the currently available (commercially) undefined liquid media only allow limited growth of fastidious bacteria such as N. gonorrhoeae from inocula in excess of 10⁴-10⁶ cfu/ml.

A range of chemically-defined liquid media are commercially available for culturing fastidious bacteria such as N. gonorrhoeae. However, none of these defined liquid media allows multiplication of fastidious bacteria from inocula of less than 10⁴-10⁶ cfu/ml.

Gonorrhoea, caused by N. gonorrhoeae, is one of the most prevalent sexually transmitted diseases worldwide. 600,000 new gonorrhoeal infections are reported annually in the United States, and in much of western Europe, rates approximate those in the US. However, the highest incidence of gonorrhoea and its complications occurs in developing countries.

Gonorrhoea is most frequently spread during sexual contact; however, it can also be transmitted from the mother's genital tract to the newborn during birth to cause ophthalmia neonatorum and systemic neonatal infection. Infection with N. gonorrhoeae is known to lead to an increased incidence of infertility and ectopic pregnancy in women. Some strains cause an asymptomatic infection, leading to an asymptomatic carrier state.

The emergence and spread of antimicrobial resistance has rendered many antibiotics (such as penicillin, tetracyclines and ciprofloxacin) ineffective in treating gonorrhoea in many parts of the world. Furthermore, the lack of a liquid culture medium capable of supporting the growth of N. gonorrhoeae requires that antimicrobial susceptibility testing must be carried out using solid, rich media as described above, and precludes the use of traditional broth-based methods that are typically used for exploring the interactions of multiple antimicrobials against a single bacterial strain.

There is therefore a need in the art for a liquid culture medium capable of supporting the growth of a diverse range of bacteria (in particular fastidious bacteria such as N. gonorrhoeae) from low inocula, yet sufficiently robust and simple to enable reproducibility.

The present invention meets this need in the art by providing a chemically-defined liquid culture medium comprising an energy source, a carbon source, a nitrogen source, an amino acid source, a purine synthesis source, a pyrimidine synthesis source and a buffer; characterised in that the culture medium has a pH less than 7, preferably less than 6.9, most preferably about 6.8.

Reference to the pH of the culture medium means the pH of the medium prior to inoculation of bacteria (eg. fastidious bacteria such as N. gonorrhoeae).

The present inventors have found that autolysis of fastidious bacteria such as N. gonorrhoeae increases with pH and appears to be maximal at approximately pH9, and minimal at approximately pH5.

In one embodiment, the osmolarity of the culture medium is typically in the range 240-285, preferably in the range 250-280, more preferably in the range 260-280, most preferably about 277 mOsm/kg.

Reference to the osmolarity of the culture medium means the osmolarity of the medium prior to inoculation of bacteria (eg. fastidious bacteria such as N. gonorrhoeae).

The culture medium may optionally comprise a component of the urea cycle or a component of the biosynthetic pathways for arginine, proline or polyamines, such as ornithine. Said component (eg. ornithine) is preferably provided in the medium at a concentration of at least 10 μM, more preferably at least 20 μM, most preferably at least 30 μM. Preferably the component (eg. ornithine) is provided at a concentration of less than 75 μM. In one aspect, the component (eg. ornithine) is provided at a concentration of between 20 μM and 80 μM, preferably between 30 μM and 50 μM, more preferably at about 36 μM.

The culture medium may optionally comprise lactate. In this regard, lactate is a useful source of carbon and energy for bacteria, in particular for fastidious bacteria such as N. gonorrhoeae. The lactate is preferably provided in the medium at a concentration of at least 5 mM, more preferably at least 7 mM, most preferably at least 8 mM. Preferably the lactate is provided at a concentration of less than 20 mM. In one aspect the lactate is provided at a concentration of about 9 mM.

The culture medium may optionally comprise ammonium bicarbonate. In this regard, ammonium bicarbonate acts as a source of nitrogen and CO₂ for bacteria (including fastidious bacteria such as N. gonorrhoeae) and as a buffer. The ammonium bicarbonate is preferably provided in the medium at a concentration of at least 10 mM, more preferably at least 12 mM, most preferably at least 15 mM. Preferably the ammonium bicarbonate is provided at a concentration of less than 35 mM. In one aspect, the ammonium bicarbonate is provided at a concentration of about 17 mM.

Suitable buffers include phosphate, acetate, bicarbonate and citrate buffers such as sodium bicarbonate buffers. However, sodium bicarbonate buffers are not preferred. Thus, in a preferred embodiment, the culture medium of the present invention does not comprise sodium bicarbonate. In this regard, the present Applicant has observed improved growth using an ammonium bicarbonate buffer compared with an equimolar concentration of sodium bicarbonate buffer.

In particular, the present Applicant has observed good growth of fastidious bacteria using an ammonium bicarbonate buffer and a pH of between 6.6 and 7.0, preferably about 6.8.

The culture medium may optionally comprise an osmoprotectant compound. Examples of suitable osmoprotectant compounds include polycationic polyamines such as spermidine. Spermidine is also important in protein and DNA synthesis. The osmoprotectant (eg. spermidine) is preferably provided in the medium at a concentration of at least 300 μM, more preferably at least 400 μM, most preferably at least 450 μM. Preferably, the osmoprotectant (eg. spermidine) is provided in the medium at a concentration of less than 1000 μM. In one aspect, the osmoprotectant (eg. spermidine) is provided at a concentration of about 455 μM.

Preferably, the culture medium is able to proliferate fastidious bacteria such as N. gonorrhoeae from an inoculum containing fewer than 10⁴ cfu/ml, preferably fewer than 10³ cfu/ml, preferably fewer than 500 cfu/ml, preferably fewer than 200 cfu/ml, preferably fewer than 100 cfu/ml, preferably fewer than 50 cfu/ml, more preferably fewer than 20 cfu/ml, most preferably about 10 cfu/ml.

The present invention also provides a chemically-defined liquid culture medium comprising an energy source, a carbon source, a nitrogen source, an amino acid source, a purine synthesis source, a pyrimidine synthesis source and a buffer; characterised in that the culture medium has an osmolarity in the range 260-280, most preferably about 277 mOsm/kg.

The present invention also provides a chemically-defined liquid culture medium comprising an energy source, a carbon source, a nitrogen source, an amino acid source, a purine synthesis source, a pyrimidine synthesis source and a buffer; characterised in that the culture medium comprises a component of the urea cycle or a component of the biosynthetic pathways for arginine, proline or polyamines, such as ornithine.

The present invention also provides a chemically-defined liquid culture medium comprising an energy source, a carbon source, a nitrogen source, an amino acid source, a purine synthesis source, a pyrimidine synthesis source and a buffer; characterised in that the culture medium comprises lactate.

The present invention also provides a chemically-defined liquid culture medium comprising an energy source, a carbon source, a nitrogen source, an amino acid source, a purine synthesis source, a pyrimidine synthesis source and a buffer; characterised in that the culture medium comprises ammonium bicarbonate.

The present invention also provides a chemically-defined liquid culture medium comprising an energy source, a carbon source, a nitrogen source, an amino acid source, a purine synthesis source, a pyrimidine synthesis source and a buffer; characterised in that the culture medium comprises an osmoprotectant compound, such as spermidine.

The present invention also provides a chemically-defined liquid culture medium comprising an energy source, a carbon source, a nitrogen source, an amino acid source, a purine synthesis source, a pyrimidine synthesis source and a buffer; characterised in that the culture medium is able to proliferate fastidious bacteria from an inoculum containing fewer than 10⁴ cfu/ml, preferably fewer than 10³ cfu/ml, preferably fewer than 500 cfu/ml, preferably fewer than 200 cfu/ml, preferably fewer than 100 cfu/ml, preferably fewer than 50 cfu/ml, more preferably fewer than 20 cfu/ml, most preferably about 10 cfu/ml.

Because the culture medium is chemically-defined and is not produced using biological extracts, the culture medium is typically substantially free of protein.

Preferably, the culture medium is substantially transparent (preferably ‘crystal clear’) and substantially colourless. Substantially transparent and colourless media are ideally suited to monitoring growth by turbidometric means (eg. using a standard laboratory spectrophotometer), including automated monitoring of growth by optical density change. Substantially transparent and colourless media are also ideally suited to detection assays that involve a colour-change, such as biochemical assays for detecting a metabolic product of a fastidious organism.

Preferably, the culture medium advantageously exhibits low background auto-fluorescence. Culture media having low fluorescence are particularly suited for use in fluorescence assays—for example, assays for detecting the presence of macromolecules, such as DNA, that have been secreted by the cultured bacteria into the medium. In this regard, the culture medium of the present invention is preferably substantially free (most preferably free) of macromolecules such as proteins and nucleic acids.

The culture medium may further comprise a vitamin/energy supplement such as nicotinamide adenine dinucleotide (NAD), ascorbic acid, biotin, calciferol, choline, folic acid, vitamin K, myo-inositol, niacinamide, nicotinic acid, pantothenic acid, pyridoxal, pyridoxine, retinol, riboflavin and tocopherol. NAD is preferably provided in the medium at a concentration of at least 5 μM, more preferably at least 8 μM, most preferably at least 9 μM. Preferably, the NAD is provided in the medium at a concentration of less than 20 μM. In one aspect, the NAD is provided at a concentration of about 9.7 μM.

Some components of the culture medium may be present in excess for ease of rapid production/reproducibility in the laboratory.

Examples of suitable energy sources for use in the above-described culture medium include glucose and lactate.

The energy source(s) is preferably included in the medium at a concentration of at least 30 mM, more preferably at least 40 mM, most preferably at least 45 mM. Preferably the energy source is present at a concentration of less than 100 mM. In one aspect, the energy source(s) is present in the medium at about 47 mM.

Examples of suitable carbon sources for use in the above-described culture medium include sugars (such as glucose), alcohols and carbohydrates.

The carbon source(s) is preferably included in the medium at a concentration of at least 20 mM, more preferably at least 30 mM, most preferably at least 35 mM. Preferably, the carbon source is present in the medium at a concentration of up to 75 mM. In one aspect, the carbon source is present at about 37 mM.

Examples of suitable nitrogen sources for use in the above-described culture medium include amino acids and ammonium bicarbonate.

The nitrogen source(s) is typically included in the medium at a concentration of at least 10 mM, more preferably at least 20 mM, most preferably at least 25 mM. Preferably, the nitrogen source is present in the medium at a concentration of up to 50 mM. In one aspect, the nitrogen source is present at about 25 mM.

Preferred amino acid sources for use in the above-described culture medium preferably include at least one of the essential amino acids—namely, tryptophan, lysine, methionine, phenylalanine, valine, leucine and isoleucine. The culture medium may also contain the amino acids histidine and/or arginine. In addition, the culture medium may contain one or more amino acid selected from asparagine, cysteine, glutamine, glycine, proline, hydroxyproline, serine, threonine and tyrosine. The amino acid source is preferably present at a concentration of at least 5 mM, preferably at least 10 mM, preferably at least 20 mM, more preferably at least 30 mM, most preferably at least 40 mM. Preferably, the amino acid source is present at a concentration of less than 50 mM. In this regard, the culture medium is preferably substantially free (most preferably free) of protein.

Examples of suitable purine synthesis sources for use in the above-described culture medium include one or more of adenine, guanine, hypoxanthine and deoxyribose. The purine synthesis source is preferably present in the medium at a concentration of at least 100 μM, preferably at least 200 μM, more preferably at least 300 μM. Preferably the purine synthesis source is present at a concentration of up to 400 μM.

Examples of suitable pyrimidine synthesis sources for use in the above-described culture medium include one or more of thymine, cytosine, uracil and deoxyribose. The pyrimidine synthesis source is preferably present in the medium at a concentration of at least 0.1 mM, preferably at least 0.25 mM, more preferably at least 0.5 mM. Preferably, the pyrimidine synthesis source is present at a concentration of up to 1.0 mM.

Examples of suitable buffers for use in the above-described culture medium include phosphate, acetate, bicarbonate and citrate buffers—eg. ammonium bicarbonate. Preferably, the culture medium does not comprise sodium bicarbonate buffer.

In one aspect, the culture medium contains at least one component selected from glutamine and oxaloacetate. In one aspect, the culture medium does not contain glutamate.

In one aspect, the culture medium is modified so that—when the medium is rendered anaerobic—the medium supports anaerobic growth of bacteria (particularly fastidious bacteria such as N. gonorrhoeae).

It is advantageous to be able to grow bacteria (particularly fastidious bacteria such as N. gonorrhoeae) under anaerobic conditions that mimic the in vivo, clinically relevant growth conditions. In this regard, facultative anaerobes such as N. gonorrhoeae may express certain proteins under anaerobic conditions that are not expressed (or only weakly expressed) under aerobic conditions. These differentially expressed proteins may be clinically useful—for example, as vaccine candidates.

It is also known that the effectiveness of many antibiotics—such as aminoglycosides (eg. gentamicin), fluoroquinolones (eg. ciprofloxacin), macrolides (eg. erythromycin) and chloramphenicol—varies under anaerobic conditions as compared with aerobic conditions. Hence, the results of susceptibility testing under aerobic conditions and anaerobic conditions may vary with respect to these antibiotics. It is therefore advantageous to be able to grow bacteria (particularly fastidious bacteria such as N. gonorrhoeae) under anaerobic conditions, in order to identify antibiotics that are clinically useful under in vivo, clinically relevant growth conditions.

The present Applicant has identified that anaerobic growth of fastidious bacteria such as N. gonorrhoeae in a culture medium of the present invention is improved by reducing the concentration of glucose in the medium. In one aspect, a reduction in the glucose content of the medium from about 10 g per 1.5 L to about 5 g per 1.5 L or less, preferably about 2 g per 1.5 L. In one aspect, the glucose content of the medium is about 5-10 mM, preferably about 7.5 mM.

The present Applicant has also identified improved anaerobic growth of fastidious bacteria (eg. N. gonorrhoeae) when the culture medium of the present invention is modified by substantially excluding lactate. In one aspect, the culture medium comprises less than 6 mM, preferably less than 5 mM, 4 mM, 3 mM, 2 mM or 1 mM lactate. Preferably, lactate is excluded from the medium.

The present Applicant has also identified improved anaerobic growth of fastidious bacteria (eg. N. gonorrhoeae) when the culture medium of the present invention is modified by substantially excluding acetate (eg. sodium acetate). In one aspect, the culture medium comprises less than 1 g sodium acetate in 1.5 L of culture medium, preferably less than 0.75 g, 0.5 g, 0.25 g sodium acetate in 1.5 L of culture medium. Most preferably, sodium acetate is excluded from the medium.

Methods for rendering a liquid culture medium anaerobic are known in the art. By way of example, it is known to add nitrite to liquid culture media. However, nitrite is carcinogenic and difficult to dispose of. Furthermore, nitrite may induce a stress response in bacteria which could slow bacterial growth. Thus, addition of nitrite is not preferred, and in one embodiment the medium is (substantially) free of nitrite.

An alternative option would be to flush the liquid culture medium with an inert gas such as nitrogen. Thus, in one aspect, the present invention provides a culture medium as described above that has been rendered substantially anaerobic (preferably anaerobic) by flushing with an inert gas.

Alternatively (or in addition) a composition may be added to the medium to render it anaerobic. Preferably, the composition keeps oxygen form intruding into the liquid culture medium and removes oxygen from the trapped air space inside a sealed container comprising culture medium.

Examples of suitable compositions include reducing agents and oxygen absorbing/scavenging agents. In one aspect, composition comprises a palladium catalyst. In one aspect, the composition may comprise an enzyme such as a mono and/or di-oxygenase. In one aspect, the composition comprises succinate. In one aspect, the composition comprises a commercially available enzyme additive Oxyrase® for Broth (Oxyrase, Inc., Mansfield, Ohio, USA).

Thus, in one aspect, the present invention provides a culture medium as described above that has been rendered substantially anaerobic (preferably anaerobic) by the addition of a composition as described above. In one aspect, the culture medium comprises said oxygen-reducing/removing agent at a concentration of about 1 mL per 50 mL of culture medium.

In one aspect, the culture medium of the present invention comprises one or more antimicrobials for certain bacteria, including fastidious bacteria, for example N. gonorrhoeae.

In one aspect, the present culture medium may comprise an indicator/reagent and/or a substrate to demonstrate enzyme activity, useful for identifying bacteria cultured therein.

The present invention also provides a method of culturing bacteria, comprising inoculating bacteria into a culture medium of the present invention.

In one embodiment, the bacteria are fastidious bacteria. Preferably, the bacteria are N. gonorrhoeae.

Preferably, the starting inoculum (eg. of fastidious bacteria such as N. gonorrhoeae) contains fewer than 10⁴ cfu/ml, preferably fewer than 10³ cfu/ml, preferably fewer than 500 cfu/ml, preferably fewer than 200 cfu/ml, preferably fewer than 100 cfu/ml, preferably fewer than 50 cfu/ml, more preferably fewer than 20 cfu/ml, most preferably about 10 cfu/ml.

When inoculated with low numbers of bacteria, including fastidious bacteria such as N. gonorrhoeae, the culture medium of the present invention typically permits proliferation to at least 10⁷ cfu/ml, preferably to at least 10⁸ cfu/ml, most preferably to at least 10⁹ cfu/ml within about 25 hours of inoculation.

Thus, in one aspect, the culture medium of the present invention is useful for growing bacteria—in particular fastidious bacteria such as N. gonorrhoeae—to high density using a single colony, or portion of a colony, from a solid medium as inoculum.

The culture medium of the present invention is also useful for growing bacteria, particularly fastidious bacteria such as N. gonorrhoeae, to high density in large volumes.

Thus, in one aspect, the culture medium is useful for growing bacteria for whole cell vaccine production or for harvesting of cell components for other purposes. In this regard, the culture medium is advantageously of low immunogenicity. For example, the culture medium is preferably substantially protein-free.

The culture medium is also useful for studying the effects of various additives or altered conditions (such as pH, atmosphere, temperature, osmolarity) on the growth of bacteria, in particular fastidious bacteria such as N. gonorrhoeae.

The physical conditions of the culture medium, such as pH or temperature, may therefore be adjusted to render it selective for organisms that are able to grow under these specific conditions.

The absence in a chemically-defined medium of macromolecules (eg. peptides, proteins, lipids) derived from the extracts, infusates or digests used to prepare traditional undefined media, ensures that such complex molecules present in the medium after growth of a bacterium are solely the synthetic products or by-products of that bacterium. These molecules may be identified or characterised by, for example, Matrix-Assisted Laser Desorption/Ionization Time-of-Flight (MALDI-TOF) mass spectrometry. MALDI-TOF (or similar spectrometry techniques) may be used to study the metabolism, physiology or pathogenicity of a bacterium; to identify bacteria, or type them for epidemiological purposes.

The culture medium may be used to identify bacteria cultured therein, particularly fastidious bacteria such as N. gonorrhoeae. In this regard, because it is chemically-defined and preferably protein/peptide-free, the culture medium enables identification of substances produced during bacterial growth that are unique to a particular species, or specific strain, of bacteria (eg. N. gonorrhoeae)—thereby enabling identification of the cultured bacteria. By way of example, an automated spectophotometric technique such as MALDI-TOF can identify ‘signature’ molecules in fluids. Because the culture medium of the present invention is chemically-defined, the un-inoculated culture medium may advantageously be used as a negative control for these investigations.

In one aspect, the culture medium may be used to investigate quorum sensing in bacteria, particularly in fastidious bacteria such as N. gonorrhoeae. In this regard, because it is chemically-defined and preferably protein/peptide-free, the culture medium enables identification of quorum-sensing signals (for example using an automated spectophotometric technique such as MALDI-TOF). The un-inoculated culture medium may advantageously be used as a negative control for these investigations.

The culture medium may also be used in fluorescence assays for identification of macromolecules such as DNA secreted from the cultured bacteria. In this regard, because the culture medium of the present invention is substantially free of macromolecules (and preferably does not contain any macromolecules), the culture medium of the present invention advantageously exhibits low background auto-fluorescence.

When supplemented with an appropriate indicator/reagent (and/or with the addition after bacterial growth of substrates to demonstrate enzyme activity), the present culture medium is also useful for identifying bacteria cultured therein.

Thus, in one aspect, the present culture medium may be used for biochemical identification of a bacterium, wherein said medium comprises an indicator to confirm the presence of said bacterium. By way of example, bacteria may be identified by detecting enzyme or other biochemical reactions important for the identification of the bacteria, especially fastidious bacteria such as N. gonorrhoeae. Preferably, the medium is (substantially) transparent and colourless, and thus ideally suited to demonstrating the colour changes typically used in clinical laboratories as end-points for such tests.

The present culture medium is also useful as a suspension medium in combination with indicators/enzyme substrates for biochemical identification tests for identifying bacteria, including fastidious bacteria such as N. gonorrhoeae.

In one aspect, the present culture medium may be used as a clinical enrichment medium for recovering bacteria, in particular fastidious bacteria such as N. gonorrhoeae, H. influenzae and S. pneumoniae in low numbers from sterile fluids (for example, joint, ascites fluid). In this regard, currently available enrichment broths do not support enrichment of fastidious bacteria such as N. gonorrhoeae.

By way of example, a volume of the culture medium is inoculated—using aseptic technique—with an aliquot of an appropriate clinical sample (such as CSF, ascites, synovial fluid) in order to identify the presence of bacteria, in particular fastidious bacteria such as N. gonorrhoeae in that sample.

Optionally, the culture medium of the present invention may be used to produce a selective medium. A selective medium comprises one or more “selection agents”—ie. components that inhibit or prevent the growth of certain species of bacteria and/or promote the growth of desired species—thereby allowing the growth of one species from a mixed inoculum.

Thus, the culture medium may further comprise one or more selection agents—typically, antimicrobials such as antibiotics.

By way of example, antibiotics such as vancomycin, colistin, amphotericin B, nystatin or trimethoprim may be added to the culture medium. In this regard, antibiotics such as vancomycin inhibit the growth of Gram-positive bacteria, antibiotics such as colistin inhibit Gram-negative bacteria, antibiotics such as amphotericin B and nystatin inhibit fungi, and antibiotics such as trimethoprim prevent the swarming of Proteus spp. These antibiotics are useful for inhibiting bacteria from sites that have normal flora present—for example, vagina, cervix, urethra—that would compete with and overgrow fastidious bacteria such as N. gonorrhoeae.

Antimicrobials for certain bacteria, including fastidious bacteria such as N. gonorrhoeae may be incorporated into the culture medium of the present invention at a range of predetermined concentrations, in order to determine the minimum inhibitory concentration (MIC) and, with subculture at 24 h, the minimum bactericidal concentration (MBC) of these antimicrobials.

Thus, in one aspect, the present culture medium is useful for testing the susceptibility of bacteria to one or more antibacterial compounds—for example, for determining the minimum inhibitory concentration (MIC) or minimum bactericidal concentration (MBC) of said antibacterial compound.

The culture medium may also be used (with an appropriate inoculum) to determine and quantify the interactions (eg. synergistic effect) of two antibacterials in time-kill or checkerboard studies. Preferably, the culture medium is (substantially) transparent and colourless, thus enabling growth to be followed turbidometrically.

Thus, in one aspect, the present culture medium is useful for enriching N. gonorrhoeae from a mixed bacterial population known to comprise N. gonorrhoeae, wherein said culture medium further comprises an antibiotic.

In one aspect, the culture medium of the present invention may be modified, as discussed above, to improve anaerobic growth of bacteria therein (ie. once the culture medium has been rendered substantially anaerobic, preferably anaerobic).

Thus, in one aspect, the present invention provides a method for culturing bacteria (preferably fastidious bacteria such as N. gonorrhoeae) under substantially anaerobic (preferably anaerobic) conditions. Preferably, the method enables culture of bacteria under anaerobic conditions that mimic in vivo, clinically relevant growth conditions.

Thus, in one aspect, once rendered substantially anaerobic (preferably anaerobic), the present culture medium is useful for testing the susceptibility of bacteria to one or more antibacterial compounds under in vivo, clinically relevant growth conditions.

In one aspect, the present invention provides a method for identifying a peptide that is differentially expressed by bacteria (preferably fastidious bacteria such as N. gonorrhoeae) under anaerobic (in vivo, clinically relevant) growth conditions as compared to aerobic growth conditions. The identified peptide could be investigated further (eg. protection studies) to determine whether it is clinically useful—eg. as a vaccine candidate.

A suitable method may comprise culturing bacteria in a culture medium of the present invention under aerobic conditions, culturing bacteria in a culture medium of the present invention that has been rendered anaerobic, and identifying a peptide that is differentially expressed under the aerobic conditions as compared to the anaerobic conditions. By way of example, the peptide may be expressed under anaerobic conditions but not expressed (or expressed at a lower level) under aerobic conditions. Alternatively, the peptide may be expressed under aerobic conditions but not expressed (or expressed at a lower level) under anaerobic conditions.

The present invention also provides a method of manufacturing a culture medium of the present invention, comprising combining an energy source, a carbon source, a nitrogen source, an amino acid source, a purine synthesis source, a pyrimidine synthesis source and a buffer. The buffer is preferably added prior to the addition of any further components, in order to reduce precipitation. In one aspect, the buffer is selected from ammonium bicarbonate and/or sodium acetate. Ammonium bicarbonate buffer is preferred.

The method may comprise adding a vitamin/energy management supplement, such as NAD. A component of the urea cycle, or a component of the biosynthetic pathways for arginine, proline or polyamines, such as ornithine, may be added. Lactate and/or ammonium bicarbonate may also be added. The method may also comprise adding an osmoprotectant compound such as a polycationic polyamine, for example, spermidine.

In one aspect, the method comprises adjusting the pH of the culture medium to pH less than 7, preferably less than 6.9, most preferably about 6.8.

In one aspect, the method comprises the step of adjusting the osmolarity of the medium to fall in the range 240-285 mOsm/kg, preferably in the range 250-280 mOsm/kg, more preferably in the range 260-280 mOsm/kg, most preferably about 277 mOsm/kg.

In one aspect, the present invention also provides a method of manufacturing a culture medium of the present invention that is suitable for anaerobic culture of bacteria (preferably fastidious bacteria such as N. gonorrhoeae). In one aspect, the culture medium is prepared as discussed above, but the glucose content is reduced as compared to the culture medium for aerobic use. In this regard, the glucose content is preferably reduced to less than 5 g per 1.5 L of culture medium, most preferably to about 2 g per 1.5 L of culture medium.

In one aspect, the culture medium for anaerobic growth is prepared as discussed above, but the lactate and/or acetate (eg. sodium acetate) content is reduced, and preferably substantially omitted (most preferably omitted).

In one aspect, the present invention provides a method for rendering a culture medium of the present invention substantially anaerobic (preferably anaerobic), comprising flushing the culture medium with an inert gas such as nitrogen.

Alternatively, or in addition, the method may comprise the step of combining the medium with a composition comprising an additive that reduces (and preferably substantially eliminates) the oxygen content of the medium. Preferably the additive creates an anaerobic environment by substantially preventing oxygen from intruding into the culture medium and/or by substantially removing oxygen from the trapped air space inside the sealed culture medium vessel. The additive may comprise a reducing agent, or an oxygen absorber or oxygen scavenger such as a palladium catalyst, or an enzyme, for example, a mono- and/or di-oxygenase, and/or succinate. One example of a suitable additive is Oxyrase for Broth® (Oxyrase, Inc., Mansfield, Ohio).

In one aspect, the method comprises adding about 1 mL of the oxygen-reducing/removing additive per 50 mL of culture medium.

The culture medium is preferably filter sterilised prior to use, for example through a 0.5 μm pore, preferably through a 0.22 μm pore.

Thus, the culture medium of the present invention may be produced relatively simply and quickly.

The present invention is discussed in more detail, by means of the Examples described below, and by the accompanying Figures, in which:

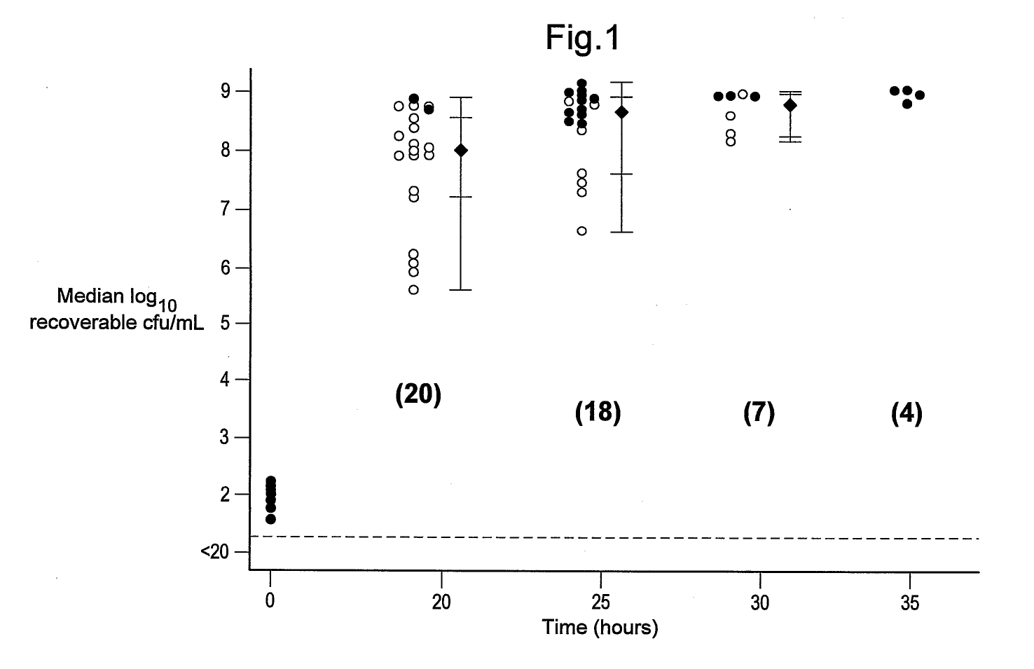

FIG. 1 illustrates the growth of 20 distinguishable strains of N. gonorrhoeae in a culture medium of the present invention.

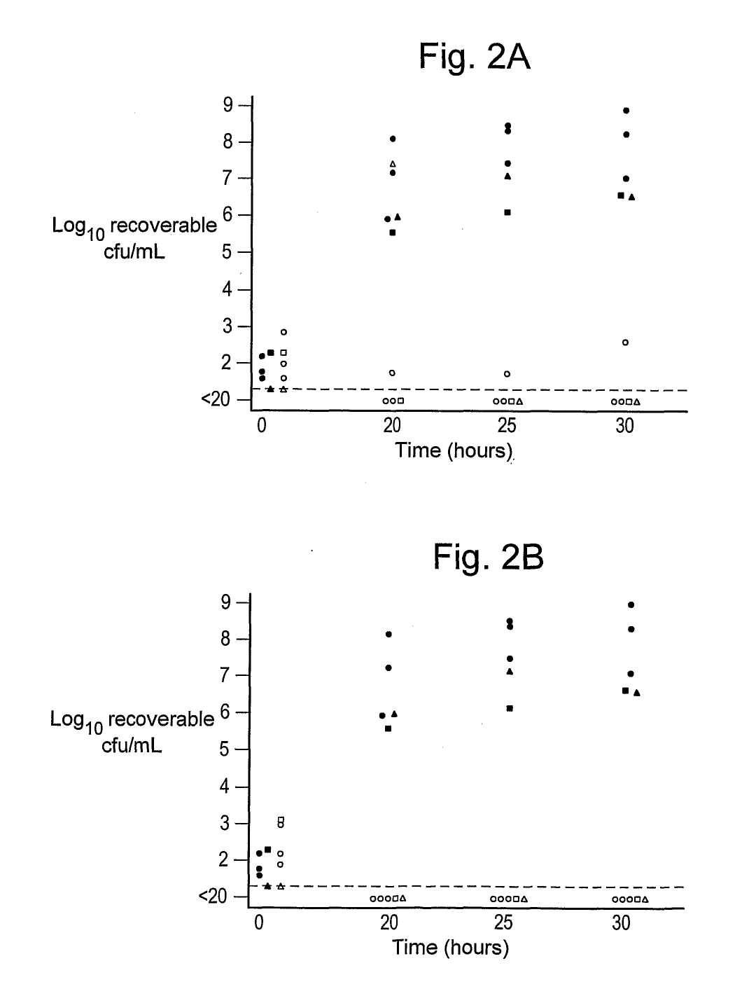

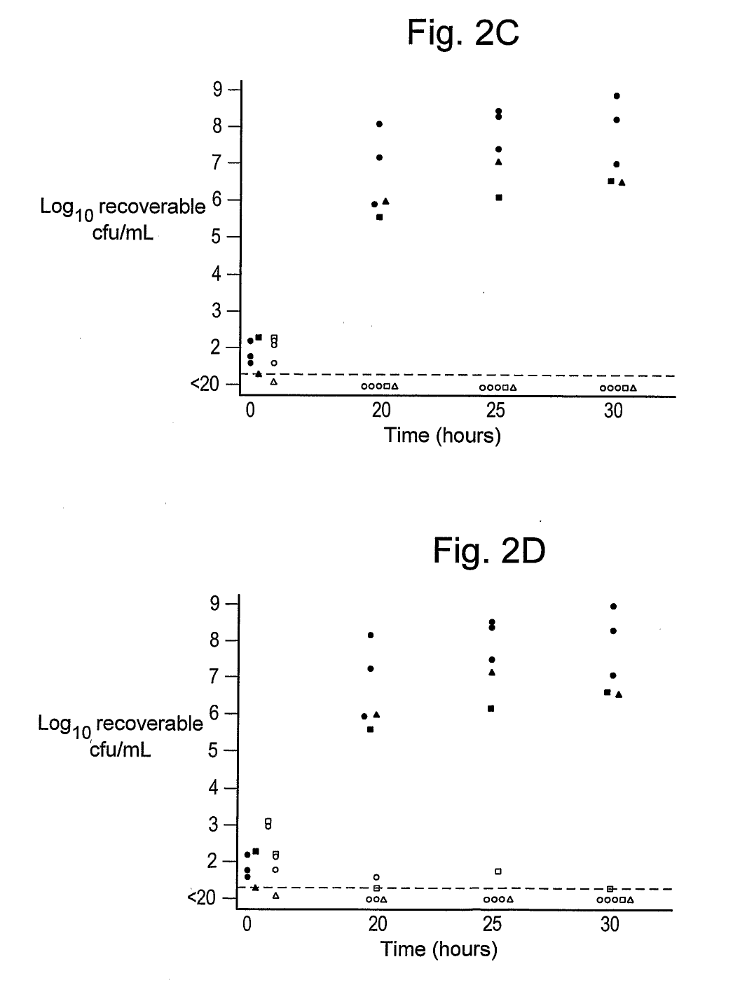

FIG. 2 illustrates the performance of the present culture medium as compared with 4 different complex media, for culture of N. gonorrhoeae, S. pneumoniae and H. influenzae.

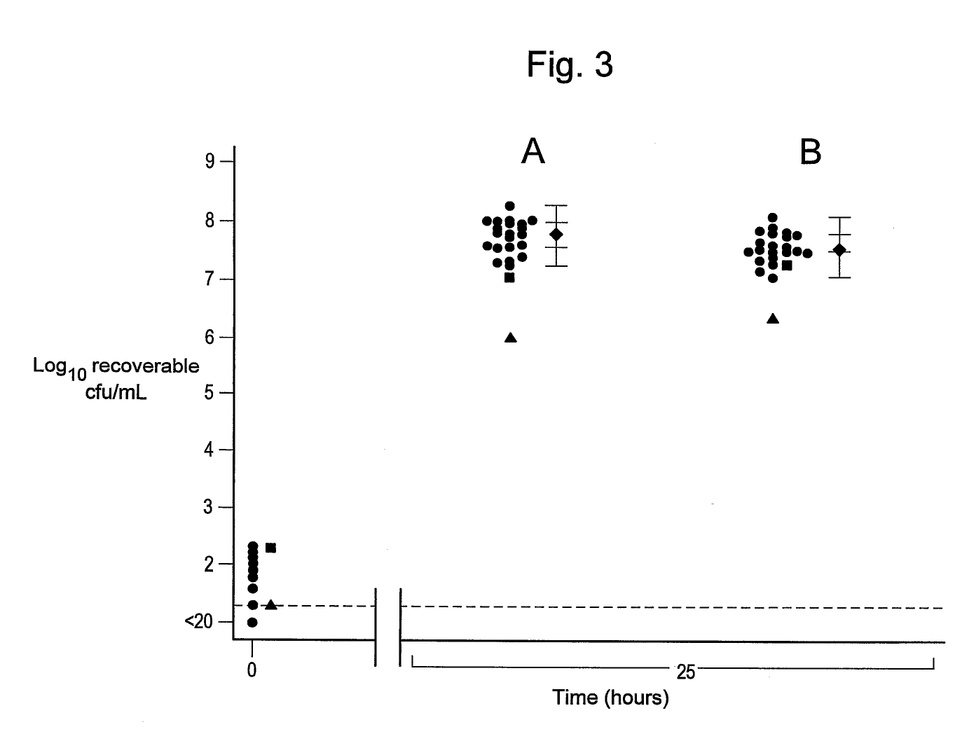

FIG. 3 illustrates the growth of 21 strains of N. gonorrhoeae, S. pneumoniae and H. influenzae in a culture medium of the present invention under conditions typical of those used in a routine clinical laboratory.

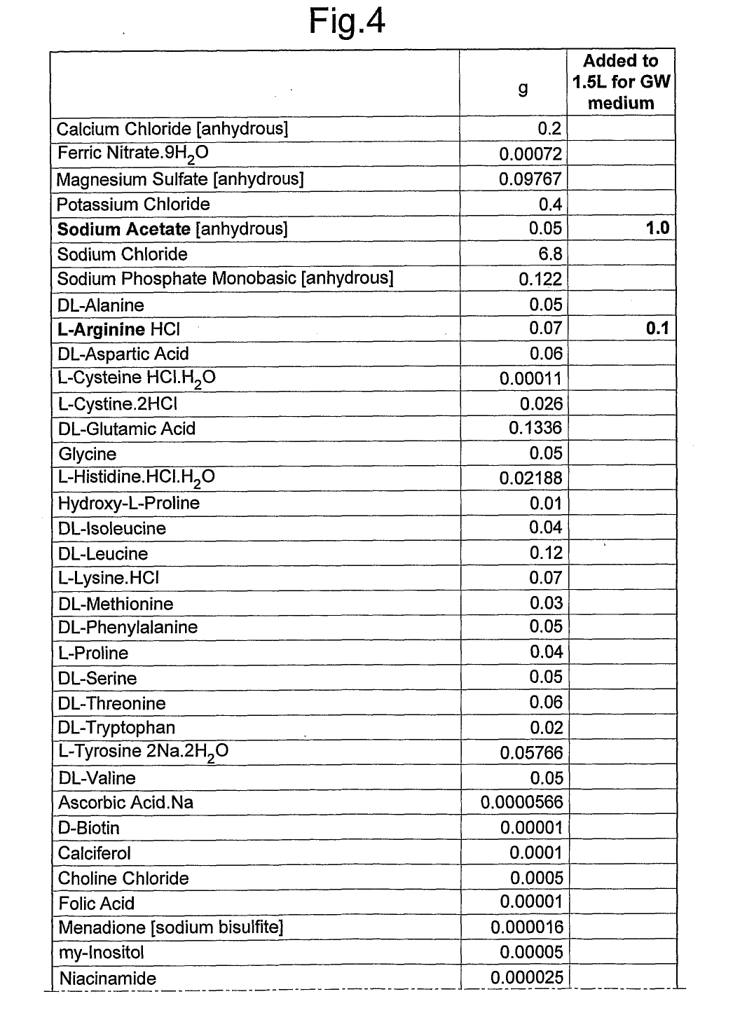

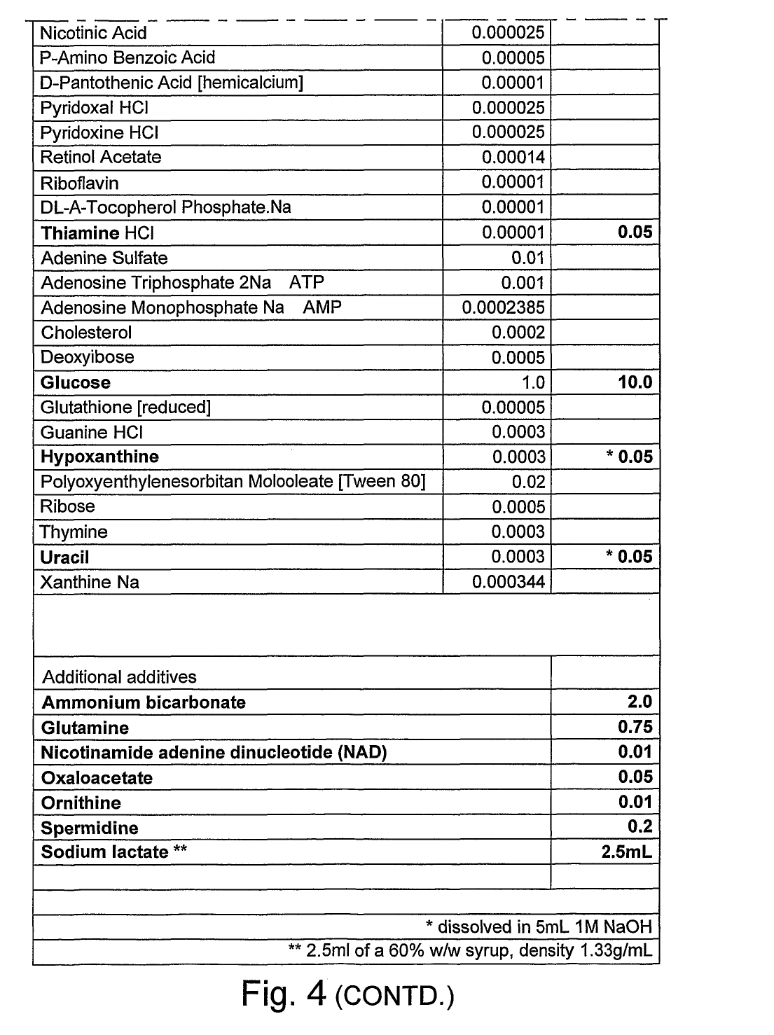

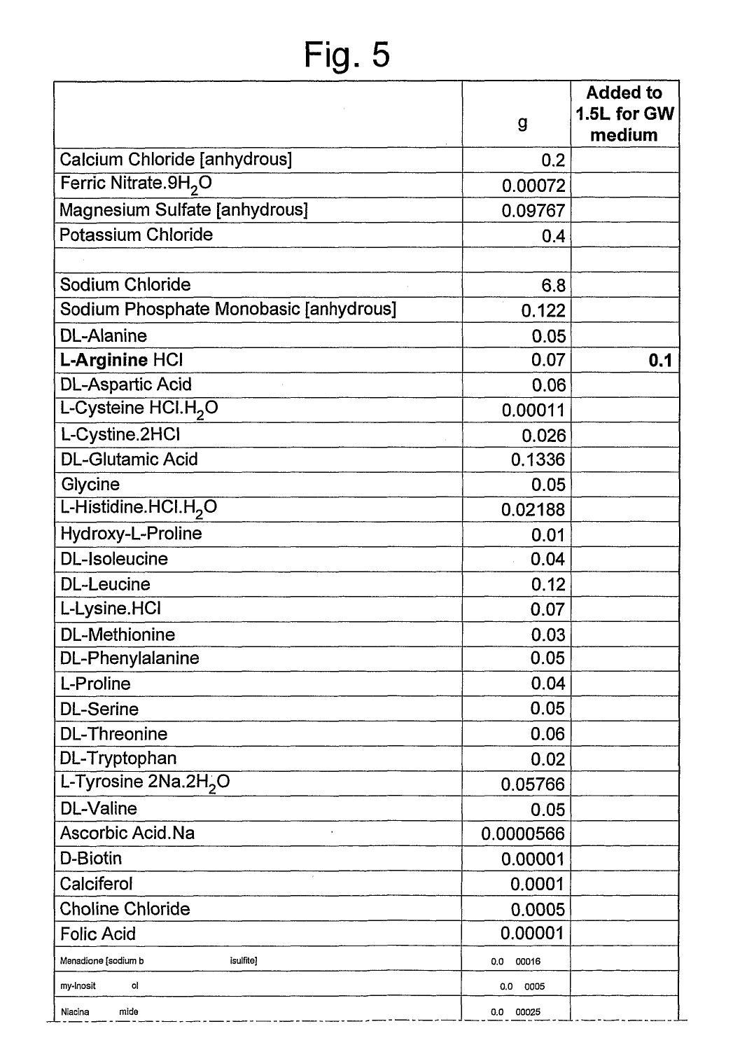

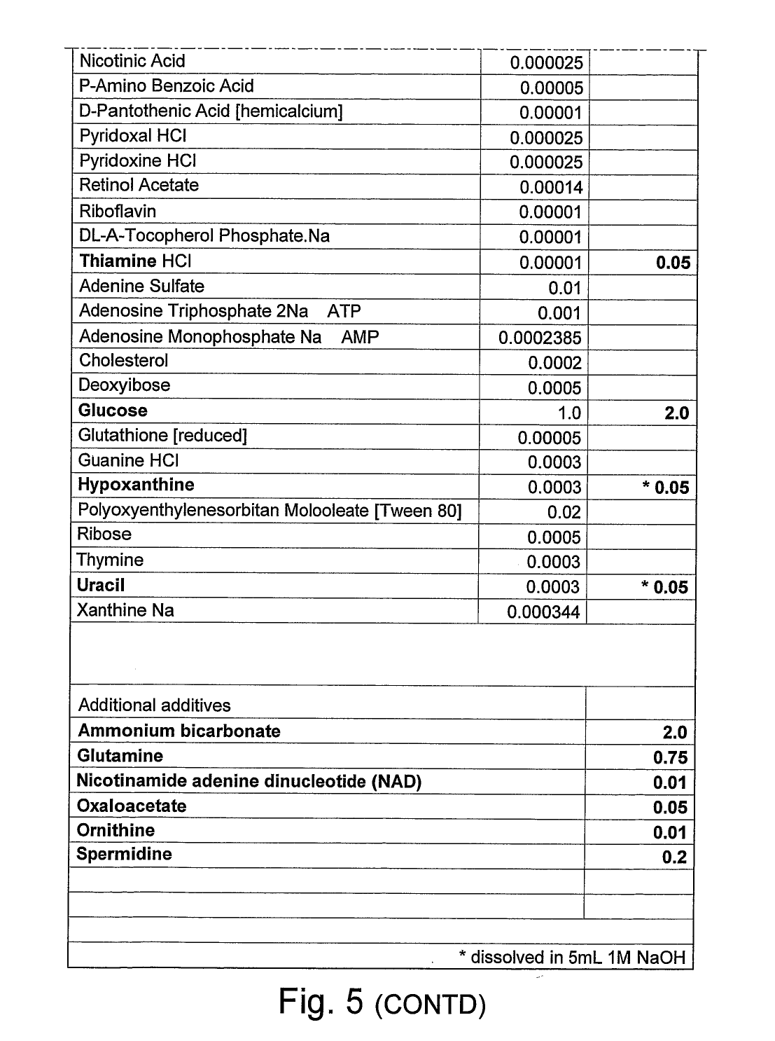

FIGS. 4 and 5 illustrate examples of specific culture media according to embodiments of the present invention.

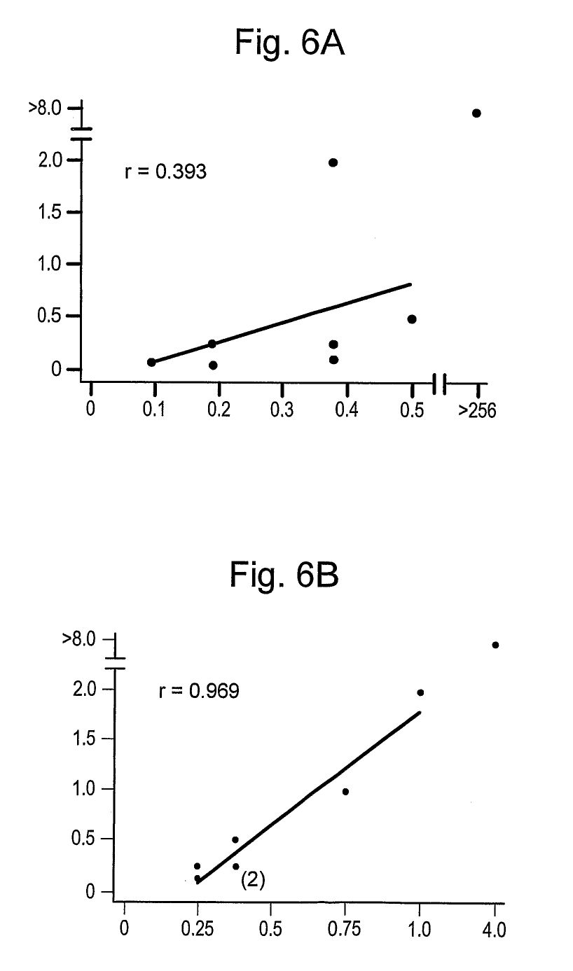

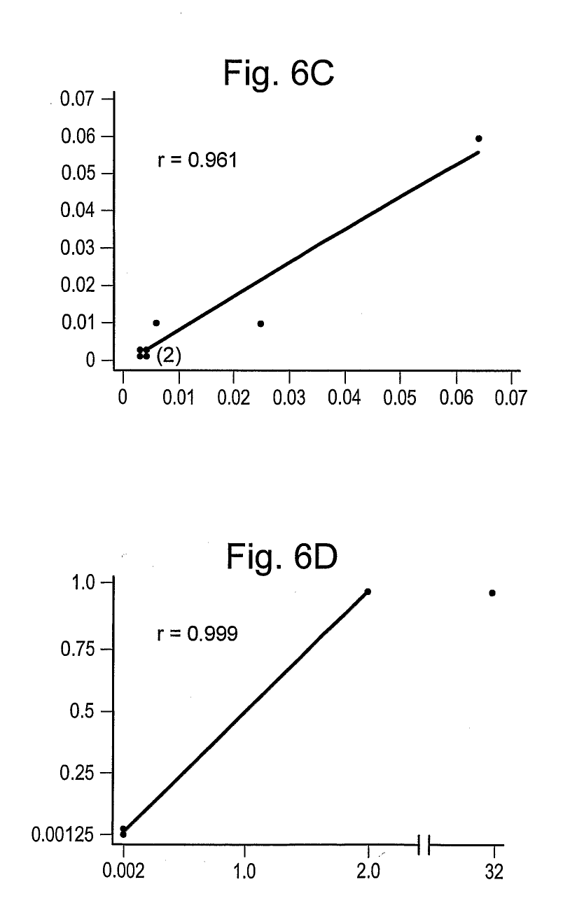

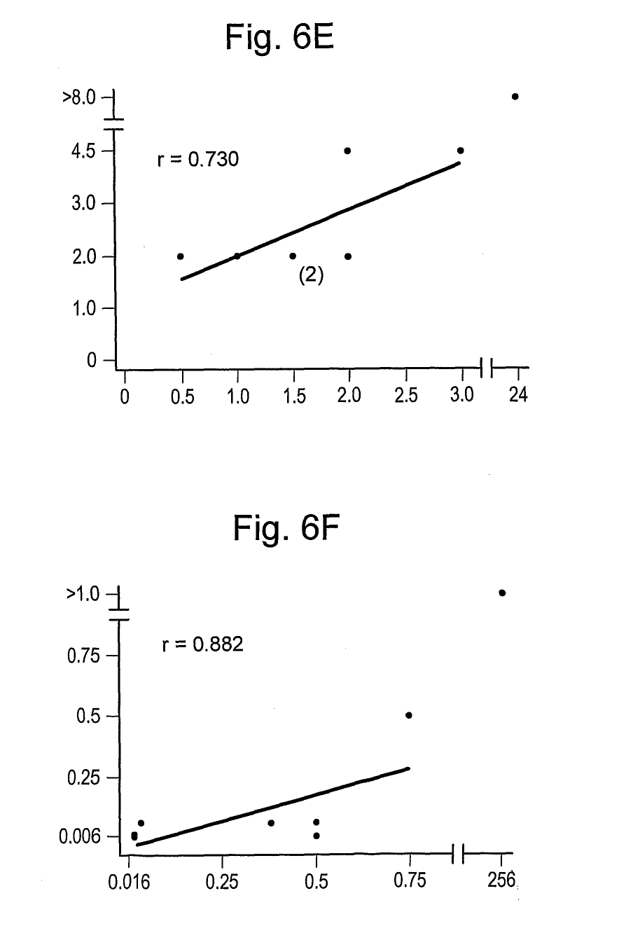

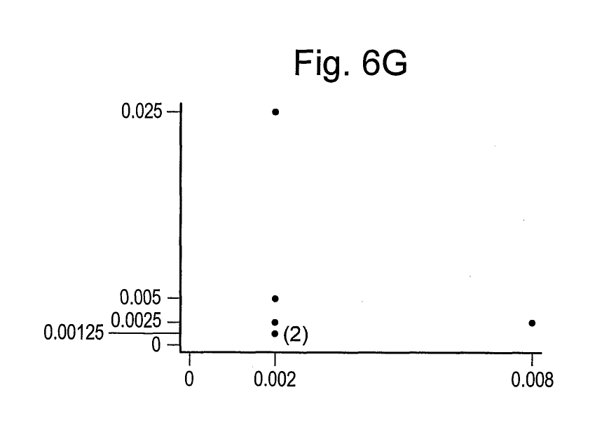

FIG. 6 illustrates the suitability of the present culture medium for determining minimum inhibitory concentrations (MICs) of antimicrobials for N. gonorrhoeae, as compared to standard E-test strips.

In more detail, FIG. 1 illustrates the growth of 20 distinguishable strains of N. gonorrhoeae in a culture medium of the present invention, under conditions allowing maximum growth of most strains. These conditions (50 mL volumes; 5% CO₂; agitation at 100 rpm) are more suited to the R&D laboratory setting (eg. when maximum yield is required rather than identification of the presence of N. gonorrhoeae) than the routine clinical laboratory. In more detail, the numbers in brackets represent the number of viable strains present at each time point. The filled points represent the maximum counts. The box-and-whisker plots show the median, IQR and range. The limit of detection (illustrated by the dotted line) is 20 cfu/ml. FIG. 1 illustrates that the median maximum yield from 25 hours onwards is typically between 10⁸ and 10⁹ cfu/mL.

FIG. 2 illustrates the performance of a culture medium of the present invention (under the same conditions as per FIG. 1) compared with three frequently used complex enrichment broths and a fourth broth that is considered to be an international standard for antimicrobial susceptibility testing: Mueller Hinton broth. In more detail, FIG. 2A compares the present culture medium with nutrient broth; FIG. 2B compares the present culture medium with tryptone soya broth; FIG. 2C compares the present culture medium with Mueller Hinton broth; and FIG. 2D compares the present culture medium with brain heart infusion. In each graph, the shaded symbols represent the culture medium of the present invention and the open symbols represent the comparison broth. Circular symbols represent growth of N. gonorrhoeae (3 different strains), square symbols represent growth of H. influenzae (1 strain), and triangular symbols represent growth of S. pneumoniae (1 strain). The present culture medium was the only medium that permitted growth to high density of all three strains of N. gonorrhoeae inoculated at very low numbers. With the exception of S. pneumoniae (which grew rapidly and to extinction in three of the commercial broths) none of the complex broths allowed sustained growth to high counts of any strain.

FIG. 3 illustrates the growth of 21 distinguishable strains of N. gonorrhoeae (represented by shaded circles), and one strain each of H. influenza (shaded squares) and S. pneumoniae (shaded triangles), in a culture medium of the present invention, under conditions typical of those used in a routine clinical laboratory: small (5 mL) volumes and without agitation. Conditions “A”=loosely capped in CO₂, not agitated. Conditions “B”=under oil, not agitated. The box-and-whisker plots show the median, IQR and range for N. gonorrhoeae. Although the median yield in both conditions tested were lower than those seen in FIG. 1, growth was more consistent and still to high counts (eg. median 10⁷-10⁸ cfu/mL at 25 h) and adequate for growth to be detected visually. In the few cases where strains had a time 0 count below the limit of detection (20 cfu/mL, illustrated by the dashed line), each of these strains subsequently grew to at least 10⁶ cfu/mL at 20 h. Good growth under oil suggests that the medium of the present invention may be useful in ‘closed’ automated systems that are loaded by capillary action.

FIG. 4 illustrates the formulation of a specific embodiment of a culture medium of the present invention.

FIG. 5 illustrates the formulation of a specific embodiment of a culture medium of the present invention, which—once rendered substantially anaerobic—is useful for anaerobic culture of fastidious bacteria such as N. gonorrhoeae.

FIG. 6 illustrates the use of a culture medium of the present invention for determining minimum inhibitory concentrations (MICs) of 6 anti-microbials for 8 strains of N. gonorrhoeae, as compared to the use of standard E-test strips on chocolate agar. The anti-microbials tested are as follows: FIG. 6A=benzylpenicillin, FIG. 6B=amoxicillin, FIG. 6C=ceftriaxone, FIG. 6D=ciprofloxacin, FIG. 6E=tetracycline, FIG. 6F=rifampicin. FIG. 6G is an expanded view of FIG. 6D. The vertical axes represent the MIC (mg/L) determined using the culture medium of the present invention, and the horizontal axes represent the MIC (mg/L) determined using E-test strips on chocolate agar, according to manufacturer's instructions. Regression lines are shown for those paired MIC values for which one or more did not exceed the highest concentration tested. Numbers in brackets indicate overlapping points. Correlation coefficients (r) are given for each anti-microbial.

### EXAMPLES

### Example 1

### Preparation of Culture Medium

An energy source (glucose 30-100 mM or lactate 30-100 mM), a carbon source (such as glucose 20-75 mM), a nitrogen source (ammonium bicarbonate or an amino acid 10-50 mM), an amino acid source (an amino acid, preferably an essential amino acid 5-50 mM), a purine synthesis source (such as adenine, guanine, hypoxanthine or deoxyribose 100-400 μM) and a pyrimidine synthesis source (such as thymine, cytosine, uracil or deoxyribose 0.1-1.0 mM) were added to 1500 mL distilled water. Glucose and a buffer (sodium acetate and/or ammonium bicarbonate) were added early on to prevent precipitation.

The remaining components of the culture medium were added next, ensuring each was dissolved before the addition of the next.

A component of the urea cycle or a component of the biosynthetic pathways for arginine, proline or polyamines was added (ornithine 10-75 mM). Lactate was added to a concentration of 5-20 mM. Ammonium bicarbonate was added to a concentration of 10-35 mM. An osmoprotectant compound such as a polycationic polyamine was added (spermidine 300-1000 μM). A vitamin/energy management supplement was added (NAD 5-20 μM).

Uracil and hypoxanthine were each dissolved in 1M NaOH before addition.

The pH was adjusted to within the desired range (less than 7, preferably less than 6.9, most preferably about 6.8+/−0.01). The osmolarity was checked and adjusted to the desired range (240-285, preferably 250-280, more preferably 260-280, most preferably about 277 mOsm/kg).

The medium was filter sterilised and stored ready for use.

One example of a culture medium prepared according to Example 1 is provided in FIG. 4.

### Example 2

### Culture of N. gonorrhoeae

Low numbers of N. gonorrhoeae (100-200 cfu/mL suspended in phosphate-buffered saline) were inoculated into 50 mL of culture medium prepared as detailed above in Example 1 in 100 mL Erlenmeyer flasks, and incubated at 37° C. on an orbital shaker at 100 rpm in 5% CO₂.

After 25 hours, the N. gonorrhoeae had grown to approximately 10⁸-10⁹ cfu/mL, as determined by colony counts using a spiral plater.

Alternatively, growth may be followed turbidometrically, using a standard laboratory spectrophotometer.

### Example 3

### Culture of N. gonorrhoeae

N. gonorrhoeae were inoculated into 5 mL volumes and incubated without agitation in 5% CO₂ at 37° C. (loosely capped in a standard laboratory CO₂ incubator).

After 25 hours, the N. gonorrhoeae had grown from low inocula of 10²-10³ cfu/mL to 10⁷-10⁸ cfu/mL, as determined by colony counts using a spiral plater.

### Example 4

### Culture of N. gonorrhoeae

N. gonorrhoeae were inoculated into 5 mL volumes and incubated without agitation under oil at 37° C. in a standard laboratory incubator.

After 25 hours, the N. gonorrhoeae had grown from low inocula of 10²-10³ cfu/mL to 10⁷-10⁸ cfu/mL, as determined by colony counts using a spiral plater.

### Example 5

### Determination of Minimum Inhibitory and Minimum Bactericidal Concentrations (MICs and MBCs) of Antimicrobials for N. gonorrhoeae

Eight clinical isolates of N. gonorrhoeae distinguishable by auxotyping and/or prior disc susceptibility testing results were studied.

Using similar conditions as described above in Examples 2, 3 or 4, incorporation of antimicrobials at a range of predetermined concentrations allows determination of the MIC (and, with subculture at 24 h, the MBC) of antibacterials for N. gonorrhoeae.

MICs were determined by both a standard macrobroth dilution method using the medium. Inoculating 2 mL volumes of the medium in test tubes with approximately 10⁵ cfu of N. gonorrhoeae produced, after overnight incubation, visible growth in the medium. Visible growth is a prerequisite for the use of such media for determining the MICs of antibiotics incorporated in the medium either in macro-broth or micro-broth methodologies or in checkerboard studies of antimicrobial combinations. Achieving visible growth confirms that turbidometric monitoring of growth is possible.

MICs were also determined using E-test strips on chocolate agar (E-test strips are widely used for MIC determinations in clinical laboratories and, for N. gonorrhoeae, the results correlate well with the gold standard agar incorporation method).

The following clinically significant antimicrobials were tested: benzylpenicillin, amoxicillin, ceftriaxone, ciprofloxacin, tetracycline and rifampicin. For each agent and both methods, MICs were categorized as susceptible, intermediate or resistant using published breakpoints. Correlation coefficients were determined (using STATA software), excluding MIC pairs where one or both values were above the highest concentration tested.

The results are illustrated in FIG. 6. There was good agreement between the two methods, expressed as correlation coefficient/percentage of strains allocated to same susceptibility category by both methods: benzylpenicillin: 0.393/75; amoxicillin: 0.969/87.5; ceftriaxone 0.961/100; ciprofloxacin: 0.999/100; tetracycline: 0.730/87.5, and rifampicin: 0.882/100.

These results confirm that GW medium is useful for MIC determination.

Adding two different antibacterials (under similar conditions) enables the interactions of the two antibacterials to be determined in time-kill or checkerboard studies.

### Example 6

### Biochemical Tests for the Identification of N. gonorrhoeae

Using similar conditions as described above in Example 2, 3 or 4, enzyme or other biochemical reactions important for the identification of N. gonorrhoeae may be identified by supplementing the culture medium with appropriate reagents (and/or with addition after growth of substrates to demonstrate enzyme activity). Since the culture medium is crystal clear and colourless, it is ideally suited to demonstrating the colour changes typically used in clinical laboratories as end-points for such tests. Such colour changes may be due to the inclusion—or addition after incubation—of a pH indicator (eg. neutral red, phenol red, bromothymol blue etc), or may follow the addition of chromogenic enzyme substrates intended to demonstrate the presence of bacterial enzymes.

### Example 7

### Preparation of ‘Anaerobic’ Culture Medium

Culture medium was prepared as described above (Example 1), with the following modifications.

These modifications provide a medium that permits, when oxygen is excluded, the growth of N. gonorrhoeae under strictly anaerobic conditions.

For this purpose the amount of added glucose is reduced, and is in the range 1-5 g per 1.5 L of final medium, typically about 2 g per 1.5 L of final medium. Both sodium acetate and lactate are excluded. The pH was adjusted to within the desired range (less than 7, preferably less than 6.9, most preferably about 6.8+/−0.01).

The oxygen content of the medium was reduced (and preferably removed from the medium) by a method selected from: (1) flushing with inert gas such as nitrogen, or (2) addition of an additive that creates an anaerobic environment—for example by preventing oxygen from intruding into the culture medium and/or removing oxygen from the trapped air space inside the culture medium vessel. Examples of suitable oxygen-removing/reducing additives include reducing agents such as palladium catalysts or an enzyme such as a mono- and/or di-oxygenase, and/or succinate. A commercially available additive is Oxyrase for Broth® (Oxyrase, Inc., Mansfield, Ohio). The additive is preferably added at a concentration of 1 mL per 50 mL of medium (ie. 30 mLs in 1.5 L of broth).

Anaerobic conditions are confirmed by the reduction to colourless of methylene blue (0.002 g/L) and the ability of the medium to support the growth of Bacteroides fragilis NCTC 9343 from approximately 2×10³ cfu/mL at time 0, to 1.4×10⁸ cfu/mL at 24 h and 4×10⁸ cfu/mL at 30 h (when oxygen is not excluded, counts for B. fragilis are <100 cfu/mL at 24 h and at 30 h).

In this modification of the medium—and under these confirmed anaerobic conditions—N. gonorrhoeae will (typically) grow from inocula of 7×10²-1.5×10³ cfu/mL (time 0) to 1×10⁶-1×10⁷ cfu/mL in 24 h-30 h, without agitation.

One example of a culture medium prepared according to Example 7 (prior to the oxygen-removing/reducing step) is provided in FIG. 5.

1. A chemically-defined liquid culture medium comprising an energy source, a carbon source, a nitrogen source, an amino acid source, a purine synthesis source, a pyrimidine synthesis source and a buffer;
characterised in that the culture medium has an osmolarity in the range 260-280.

2. A chemically-defined liquid culture medium according to claim 1, wherein the culture medium further comprises a component of the urea cycle or a component of the biosynthetic pathways for arginine, proline or polyamines.

3. A chemically-defined liquid culture medium according to claim 1, wherein the culture medium further comprises lactate.

4. A chemically-defined liquid culture medium according to claim 1, wherein the culture medium further comprises ammonium bicarbonate.

5. A chemically-defined liquid culture medium according to claim 1, wherein the culture medium comprises an osmoprotectant compound.

6. A chemically-defined liquid culture medium according to claim 5, wherein the said osmoprotectant compound is a polycationic polyamine.

7. A chemically-defined liquid culture medium according to claim 1, wherein the culture medium is able to proliferate fastidious bacteria from an inoculum containing fewer than 10⁴ cfu/ml.

8. A chemically-defined liquid culture medium according to claim 1 wherein the culture medium is anaerobic.

9. A chemically-defined liquid culture medium according to claim 1, wherein the culture medium has a pH less than 7.

10-46. (canceled)

47. A chemically-defined liquid culture medium according to claim 1 wherein the culture medium is substantially free of protein.

48. A chemically-defined liquid culture medium according to claim 1 wherein the culture medium is substantially colourless and/or transparent.

49. A chemically-defined liquid culture medium according to claim 1 wherein the culture medium further comprises nicotinamide adenine dinucleotide (NAD).

50. A chemically-defined liquid culture medium according to claim 1 wherein the culture medium further comprises an antibiotic.

51-72. (canceled)
